(19) United States
(12) Patent Application Publication
(10) Pub. No.: US 2025/0340665 A1
(43) Pub. Date: Nov. 6, 2025

(54) METHODS OF TREATING DERMATOMYOSITIS

(71) Applicant: ADIENNE S.A., Lugano (CH)

(72) Inventor: Antonio Francesco DI NARO, Morcote (CH)

(21) Appl. No.: 18/507,923

(22) PCT Filed: May 13, 2022
(86) PCT No.: PCT/IB2022/054500

§ 371 Date: Nov. 13, 2023

Related U.S. Application Data
(60) Provisional application No. 63/201,806, filed on May 13, 2021.

(51) Int. Cl. C07K 16/28; A61K 31/365; A61K 31/52; A61K 39/00; A61K 45/06; C07K 16/40

(52) U.S. Cl. CPC C07K 16/2896; A61K 31/365; A61K 31/52; A61K 45/06; C07K 16/40; A61K 2039/545; C07K 2317/24; C07K 2317/565

(57) ABSTRACT

This disclosure relates to methods of using an anti-CD26 antibody and antigen binding fragments thereof for the treatment of dermatomyositis.

### FIELD

This disclosure relates to methods of using an anti-CD26 antibody and antigen binding fragments thereof for the treatment of dermatomyositis.

### BACKGROUND

Dermatomyositis (DM) is a type of inflammatory myopathy characterized by inflammatory and degenerative changes of the muscles and skin. Associated symptoms and physical findings may vary widely from case to case as patients may present differently. Muscle abnormalities may begin with aches and weakness of the muscles of the trunk, upper arms, hips, and thighs (proximal muscles). Muscles may be stiff, sore, tender and, eventually, show signs of degeneration (atrophy). Affected individuals may experience difficulty in performing certain functions, such as raising their arms and/or climbing stairs or develop speech and swallowing difficulties. Skin abnormalities associated with dermatomyositis often include a distinctive reddish-purple rash (heliotrope rash) on the upper eyelid or across the cheeks and bridge of the nose in a “butterfly” distribution and on the forehead and scalp. Other characteristic rashes include scaling and redness of the knuckles, elbows, knees, and/or other extensor regions (Gottron papules and sign); an abnormal accumulation of fluid (edema) in body tissues surrounding the eyes; and/or other features. The symptoms of childhood (juvenile) dermatomyositis (JDM) are similar to those associated with the adult form of the disorder. However, onset is usually more sudden. In addition, abnormal accumulations of calcium deposits (calcifications) in muscle and skin tissues as well as involvement of the digestive tract are more common in JDM.

The treatment of dermatomyositis is directed toward the specific symptoms that are apparent in each individual and thus can vary from one patient to another. In general, treatment for the muscle involvement associated with dermatomyositis requires the use of glucocorticoids. Treatment for the skin findings associated with dermatomyositis includes: sun avoidance, sunscreens, topical glucocorticoids, anti-malarial agents, methotrexate, mycophenolate mofetil, and/or intravenous immunoglobulin (IVIg).

Glucocorticoids, particularly prednisone, are widely used in the treatment of dermatomyositis and are often used first-line. Such medications, which are similar to the natural hormones produced by the outer region of the adrenal glands, are often used to reduce inflammation and associated swelling and also serve to suppress immune responses.

High dose glucocorticoid therapy may produce adverse side effects, particularly after prolonged use, such as a decrease in bone density, causing bones to become brittle and weakened (osteoporosis); increasing, “superimposed” muscle weakness due to effects of the medication (i.e., corticosteroid myopathy); tissue swelling (edema); peptic ulcers; elevated blood pressure; elevated blood sugar levels; weight gain with fat deposits in the abdomen, face, and/or back of the neck or other findings.

Therefore, there exists a need in the art for improved treatment methods for dermatomyositis.

### SUMMARY

The present disclosure is directed to a method of treating dermatomyositis in a subject, comprising administering a therapeutically effective amount of an anti-CD26 antibody to the subject, wherein the antibody is administered at a dose of 16 mg/m²/day, and wherein the antibody is administered once daily for five days followed by administration three times per week for a total of 16 doses.

The present disclosure is also directed to a method of reducing the levels of dermatomyositis-related cytokines in a subject, comprising administering a therapeutically effective amount of an anti-CD26 antibody to the subject, wherein the antibody is administered at a dose of 16 mg/m2/day, and wherein the antibody is administered once daily for five days followed by administration three times per week for a total of 16 doses.

In one aspect, the dermatomyositis-related cytokines are tumor necrosis factor-alpha (TNFα), interleukin-1β (IL-1β) interleukin-6 (IL-6), interleukin-17 (IL-17), interleukin-21 (IL_21) interferon-alfa (IFNα) or interferon-gamma (IFNγ).

In one aspect, the anti-CD26 antibody is a full-length antibody. In another aspect, the antibody is a monoclonal, human, humanized, chimeric, multivalent antibody, or an antigen-binding fragment thereof.

In one aspect, the antibody has an isotype selected from the group consisting of IgG1, IgG2, IgG3, IgG4, IgM, IgA, IgD and IgE. In another aspect, the antibody has an IgG2b isotype. In another aspect, the anti-CD26 antibody is begelomab, 1F7, or CM03. In another aspect, the anti-CD26 antibody is produced in Chinese hamster ovary (CHO) cells. In an other aspect, the anti-CD26 antibody is produced from a hybridoma cell line deposited at CBA-ICLC of Genoa (Italy) as deposit number PD 12002.

In one aspect, the anti-CD26 antibody comprises
(a) a heavy chain variable region CDR1 comprising the sequence set forth in SEQ ID NO:7;
(b) a heavy chain variable region CDR2 comprising the sequence set forth in SEQ ID NO:8;
(c) a heavy chain variable region CDR3 comprising the sequence set forth in SEQ ID NO:9;
(d) a light chain variable region CDR1 comprising the sequence set forth in SEQ ID NO:10;
(e) a light chain variable region CDR2 comprising the sequence set forth in SEQ ID NO:11; and
(f) a light chain variable region CDR3 comprising the sequence set forth in SEQ ID NO:12. In another aspect, the anti-CD26 antibody comprises heavy and light chain variable regions comprising the sequences set forth in SEQ ID NOs:3 and 5, respectively. In another aspect, the anti-CD26 antibody comprises heavy and light chains comprising the sequences set forth in SEQ ID NOs: 1 and 2, respectively.

In one aspect the methods of the invention further comprise administering a glucocorticoid and/or immunosuppressant therapy. In another aspect, the immunosuppressant therapy comprises administration of methotrexate, azathioprine, or mycophenolate.

### DETAILED DESCRIPTION

In order that the present disclosure may be more readily understood, certain terms are first defined. As used in this application, except as otherwise expressly provided herein, each of the following terms shall have the meaning set forth below. Additional definitions are set forth throughout the application.

### Definitions

An “antibody” (Ab) shall include, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. A heavy chain may have the C-terminal lysine or not. Unless specified otherwise herein, the amino acids in the variable regions are numbered using the Kabat numbering system and those in the constant regions are numbered using the EU system. In one embodiment, an antibody is an intact antibody.

An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, monoclonal and polyclonal antibodies; chimeric and humanized antibodies; human or nonhuman antibodies; wholly synthetic antibodies; and single chain antibodies. A nonhuman antibody may be humanized by recombinant methods to reduce its immunogenicity in man. Where not expressly stated, and unless the context indicates otherwise, the term “antibody” includes monospecific, bispecific, multivalent or multi-specific, antibodies, as well as a single chain antibody.

As used herein, an “IgG antibody” has the structure of a naturally occurring IgG antibody, i.e., it has the same number of heavy and light chains and disulfide bonds as a naturally occurring IgG antibody of the same subclass. For example, an anti-CD26 IgG1, IgG2, IgG3 or IgG4 antibody consists of two heavy chains (HCs) and two light chains (LCs), wherein the two heavy chains and light chains are linked by the same number and location of disulfide bridges that occur in naturally occurring IgG1, IgG2, IgG3 and IgG4 antibodies, respectively (unless the antibody has been mutated to modify the disulfide bonds)

An “isolated antibody” refers to an antibody that is substantially free of other antibodies having different antigenic specificities (e.g., an isolated antibody that binds specifically to CD26 is substantially free of antibodies that bind specifically to antigens other than CD26). An isolated antibody that binds specifically to CD26 may, however, have cross-reactivity to other antigens, such as CD26 molecules from different species. Moreover, an isolated antibody may be substantially free of other cellular material and/or chemicals.

The antibody may be an antibody that has been altered (e.g., by mutation, deletion, substitution, conjugation to a non-antibody moiety). For example, an antibody may include one or more variant amino acids (compared to a naturally occurring antibody) which change a property (e.g., a functional property) of the antibody. For example, numerous such alterations are known in the art which affect, e.g., half-life, effector function, and/or immune responses to the antibody in a patient. The term antibody also includes artificial polypeptide constructs which comprise at least one antibody-derived antigen binding site.

The term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A mAb is an example of an isolated antibody. MAbs may be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region is also derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized antibody” refers to an antibody in which some, most or all of the amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen” antibody refers to an antibody that binds specifically to the antigen. For example, an anti-CD26 antibody binds specifically to CD26.

An “antigen-binding portion” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments or portions of a full-length antibody. Examples of binding fragments encompassed within the term “antigen-binding portion” or “antigen-binding fragment” of an antibody, e.g., an anti-CD26 antibody described herein, include:
(1) a Fab fragment (fragment from papain cleavage) or a similar monovalent fragment consisting of the VL, VH, LC and CH1 domains;
(2) a F(ab′)2 fragment (fragment from pepsin cleavage) or a similar bivalent fragment comprising two Fab fragments linked by a disulfide bridge at the hinge region;
(3) a Fd fragment consisting of the VH and CH1 domains;
(4) a Fv fragment consisting of the VL and VH domains of a single arm of an antibody,
(5) a single domain antibody (dAb) fragment (Ward et al., (1989) Nature 341:544-46), which consists of a VH domain;
(6) a bi-single domain antibody which consists of two VH domains linked by a hinge (dual-affinity re-targeting antibodies (DARTs));
(7) a dual variable domain immunoglobulin;
(8) an isolated complementarity determining region (CDR); and
(9) a combination of two or more isolated CDRs, which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” or “antigen-binding fragment” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins. In some embodiments, an antibody is an antigen-binding fragment.

The term “CD26” refers to dipeptidyl peptidase 4 (DPP4). The terms CD26 and DPP4 are used interchangeably herein. The term “CD26” includes variants, isoforms, homologs, orthologs and paralogs. For example, antibodies specific for a human CD26 protein may, in certain cases, cross-react with a CD26 protein from a species other than human. In other embodiments, the antibodies specific for a human CD26 protein may be completely specific for the human CD26 protein and may not exhibit species or other types of cross-reactivity, or may cross-react with CD26 from certain other species, but not all other species (e.g., cross-react with monkey CD26 but not mouse CD26). The term “human CD26” refers to human sequence CD26, such as the complete amino acid sequence of human CD26 having GenBank Accession No. AH005372.3. The human CD26 sequence may differ from human CD26 of GenBank Accession No. AH005372.3 by having, e.g., conserved mutations or mutations in non-conserved regions and the CD26 has substantially the same biological function as the human CD26 of GenBank Accession No. AH005372.3.

A particular human CD26 sequence will generally be at least 90% identical in amino acid sequence to human CD26 of GenBank Accession No. AH005372.3 and contains amino acid residues that identify the amino acid sequence as being human when compared to CD26 amino acid sequences of other species (e.g., murine). In certain cases, a human CD26 can be at least 95%, or even at least 96%, 97%, 98%, or 99% identical in amino acid sequence to CD26 of GenBank Accession No. AH005372.3. In certain embodiments, a human CD26 sequence will display no more than 10 amino acid differences from the CD26 sequence of GenBank Accession No. AH005372.3. In certain embodiments, the human CD26 can display no more than 5, or even no more than 4, 3, 2, or 1 amino acid difference from the CD26 sequence of GenBank Accession No. AH005372.3.

“Percent (%) amino acid sequence identity” with respect to a polypeptide sequence as set forth herein is defined as the percentage of amino acid residues in a candidate sequence of interest to be compared that are identical with the amino acid residues in a particular polypeptide sequence as set forth herein (e.g. a particular polypeptide sequence characterized by a sequence identifier in the sequence listings), after aligning the sequences and introducing gaps, if necessary, to achieve the maximum percent sequence identity, and not considering any conservative substitutions as part of the sequence identity. A sequence alignment performed for determining percent amino acid sequence identity can be carried out according to procedures known in the art, as described for example in EP 1 241 179 B1, which is incorporated herewith by reference, including in particular page 9, line 35 to page 10, line 40 with the definitions used therein and Table 1 regarding possible conservative substitutions. For example, a skilled person can use publicly available computer software. Computer program methods for determining sequence identity include, but are not limited to BLAST, BLAST-2, ALIGN or Megalign (DNASTAR) software. According to one embodiment, the software alignment program used can be BLAST. A skilled person can determine appropriate parameters for measuring alignment, including any algorithms needed to achieve maximal alignment over the full length of the sequences subjected to comparison. According to one embodiment, the % identity values can be generated using the WU-BLAST-2 computer program (Altschul et al., 1996, Methods in Enzymology 266:460-480, which is incorporated herewith by reference). According to one embodiment, the following parameters are used, when carrying out the WU-BLAST-2 computer program: Most of the WU-BLAST-2 search parameters are set to the default values. The adjustable parameters were set with the following values: overlap span=1, overlap fraction=0.125, word threshold (T)=11, and scoring matrix=BLOSUM62. The HSP S and HSP S2 parameters, which are dynamic values used by BLAST-2, are established by the program itself depending upon the composition of the sequence of interest and composition of the database against which the sequence is being searched. However, the values can be adjusted to increase sensitivity. A % sequence identity value can be determined by dividing (a) the number of matching identical amino acid residues between a particular amino acid sequence as set forth herein which is subjected to comparison (e.g. a particular polypeptide sequence characterized by a sequence identifier in the sequence listings) and the candidate amino acid sequence of interest to be compared, for example the number of matching identical amino acid residues as determined by WU-BLAST-2, by (b) the total number of amino acid residues of the polypeptide sequence as set forth herein which is subjected to comparison (e.g. a particular polypeptide sequence characterized by a SEQ. ID. NO. in the sequence listings).

“Percent (%) nucleic acid sequence identity” with respect to a nucleic acid sequence as set forth herein is defined as the percentage of nucleotides in a candidate sequence of interest to be compared that are identical with the nucleotides in a particular nucleic acid sequence as set forth herein (e.g. a particular polypeptide sequence characterized by a sequence identifier in the sequence listings), after aligning the sequences and introducing gaps, if necessary, to achieve the maximum percent sequence identity. An alignment for purposes of determining percent nucleic acid sequence identity can be carried out according to procedures known in the art, as described for example in EP 1 241 179 B1. For example, a skilled person can use publicly available computer software, such as using publicly available computer software such as BLAST, BLAST-2, ALIGN or Megalign (DNASTAR) software. A skilled person can determine appropriate parameters for measuring alignment, including any algorithms needed to achieve maximal alignment over the full length of the sequences subjected to comparison. According to a preferred embodiment, the % identity values can be generated using the WU-BLAST-2 computer program. According to a preferred embodiment, the following computer program and parameters are used: The identity values used herein are generated by the BLASTN module of WU-BLAST-2 set to the default parameters, with overlap span and overlap fraction set to 1 and 0.125, respectively. A % nucleic acid sequence identity value can be obtained by dividing (a) the number of matching identical nucleotides between a particular nucleic acid sequence as set forth herein which is subjected to comparison (e.g. a particular nucleic acid sequence characterized by a sequence identifier in the sequence listings), and the comparison nucleic acid molecule of interest to be compared, for example the number of matching identical nucleotides as determined by WU-BLAST-2, by (b) the total number of nucleotide residues of the particular nucleic acid sequence as set forth herein which is subjected to comparison (e.g. a particular nucleic acid sequence characterized by a sequence identifier in the sequence listings).

A “patient” as used herein includes any patient who is afflicted with dermatomyositis. The terms “subject” and “patient” are used interchangeably herein.

“Administering” refers to the physical introduction of a composition comprising a therapeutic agent to a subject, using any of the various methods and delivery systems known to those skilled in the art. Routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, in some embodiments, orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

“Treatment” or “therapy” of a subject refers to any type of intervention or process performed on, or the administration of an active agent to, the subject with the objective of reversing, alleviating, ameliorating, inhibiting, slowing down progression, development, severity or recurrence of a symptom, complication or condition, or biochemical indicia associated with a disease.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system.

In one example, an “effective amount” is the amount of anti-CD26 antibody clinically proven to affect a significant decrease in the inflammatory response of dermatomyositis. As used herein, the terms “fixed dose”, “flat dose” and “flat-fixed dose” are used interchangeably and refer to a dose that is administered to a patient without regard for the weight or body surface area (BSA) of the patient. The fixed or flat dose is therefore not provided as a mg/m² dose, but rather as an absolute amount of the agent (e.g., the anti-CD26 antibody). For example, a 60 kg person and a 100 kg person would receive the same dose of the composition (e.g., 100 mg of an anti-CD26 antibody).

The term “body surface area based dose” as referred to herein means that a dose that is administered to a patient is calculated based on the surface area of the patient. For example, when a patient with 2.0 body surface area (BSA) requires 4 mg/m² of an anti-CD26 antibody, one can draw the appropriate amounts of the anti-CD26 antibody (i.e., 8 mg).

“Dosing interval,” as used herein, means the amount of time that elapses between doses of the anti-CD26 antibody disclosed herein being administered to a subject. Dosing interval can thus be indicated as ranges.

The term “dosing frequency” as used herein refers to the frequency of administering doses of the anti-CD26 antibody disclosed herein in a given time. Dosing frequency can be indicated as the number of doses per a given time, e.g., once a week or once in two weeks.

The terms “about once a week,” “once about every week,” “once about every two weeks,” or any other similar dosing interval terms as used herein means approximate number, and “about once a week” or “once about every week” can include every seven days±two days, i.e., every five days to every nine days. The dosing frequency of “once a week” thus can be every five days, every six days, every seven days, every eight days, or every nine days. “Once about every two weeks” can include every fourteen days±three days, i.e., every eleven days to every seventeen days. Similar approximations apply, for example, to once about every three weeks, once about every four weeks, once about every five weeks, once about every six weeks and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

The term “CD26 positive” or “CD26 expression positive,” relating to CD26 expression, refers to the proportion of cells in a test tissue sample, typically comprising muscle cells, which the tissue sample is scored as expressing CD26

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including antibodies, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The use of the alternative (e.g., “or”) should be understood to mean either one, both, or any combination thereof of the alternatives. As used herein, the indefinite articles “a” or “an” should be understood to refer to “one or more” of any recited or enumerated component.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one-tenth and one-hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary of Biochemistry and Molecular Biology, 2006, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

Various aspects of the invention are described in further detail in the following subsections.

### CD26 Antibodies

In one aspect, the invention features methods of using an anti-CD26 antibody in the treatment of dermatomyositis. Anti-human-CD26 antibodies (or VH/VL domains derived therefrom) suitable for use in the invention can be generated using methods well known in the art. Alternatively, art recognized anti-CD26 antibodies can be used.

In some embodiments, the anti-CD26 antibody is produced from the hybridoma cell line deposited on Sep. 11, 2012 under the Budapest Treaty at the Centro di Biotecnologie Avanzate (CBA)-Interlab Cell Line Collection (ICLC) of Genoa (L. go R. Benzi, 10, Genoa, Italy) as deposit PD 12002. In another embodiment, the anti-CD26 antibody used in the methods of the invention bind the same epitope as an antibody produced by the hybridoma cell line deposited at CBA-ICLC of Genoa (Italy) deposited as PD 12002.

In some embodiments, the anti-CD26 antibody is begelomab comprising heavy and light chains comprising the sequences shown in SEQ ID NOs:1 and 2, respectively, or antigen binding fragments and variants thereof, as described in U.S. Pat. Nos. 9,376,498 and 10,208,126, the teachings of which are hereby incorporated by reference.

In other embodiments, the antibody has the heavy and light chain CDRs or variable regions of begelomab. Accordingly, in one embodiment, the antibody comprises CDR1, CDR2, and CDR3 domains of the VH region of begelomab having the sequence set forth in SEQ ID NO:3, and CDR1, CDR2 and CDR3 domains of the VL region of begelomab having the sequence set forth in SEQ ID NO:5. In another embodiment, the antibody comprises CDR1, CDR2 and CDR3 domains comprising the sequences set forth in SEQ ID NOs:7, 8, and 9, respectively, and CDR1, CDR2 and CDR3 domains comprising the sequences set forth in SEQ ID NOs:10, 11, and 12, respectively. In another embodiment, the antibody comprises VH and/or VL regions comprising the amino acid sequences set forth in SEQ ID NO:3 and/or SEQ ID NO: 5, respectively. In another embodiment, the antibody comprises heavy chain variable (VH) and/or light chain variable (VL) regions encoded by the nucleic acid sequences set forth in SEQ ID NO:4 and/or SEQ ID NO:6, respectively. In another embodiment, the antibody competes for binding with and/or binds to the same epitope on CD26 as the above-mentioned antibodies. In another embodiment, the antibody has at least about 90% variable region amino acid sequence identity with the above-mentioned antibodies (e.g., at least about 90%, 95% or 99% variable region identity with SEQ ID NO:3 or SEQ ID NO:5).

In some embodiments, the anti-CD26 antibody or antigen-binding portion thereof cross-competes with begelomab for binding to human CD26. In other embodiments, the anti-CD26 antibody or antigen-binding portion thereof binds to the same epitope as begelomab.

In some embodiments, the anti-CD26 antibody is codon-optimized for expression in a host cell. In one embodiment, the anti-CD26 antibody is begelomab that is codon optimized for expression in Chinese Hamster Ovary (CHO) cells. In another embodiment, the codon optimized begelomab comprises the heavy chain and light chain of SEQ ID NOs: 15 and 16, respectively.

In one embodiment, the anti-CD26 antibody is an antibody or antigen-binding fragment thereof described in U.S. Pat. No. 7,658,923 (for example, 1F7); U.S. Pat. No. 7,462,698 (for example, CM03), U.S. Pat. No. 8,771,688, and EP Pat. No. 3 348 276 A1. Antibodies or antigen-binding fragments thereof that compete with any of the above-referenced art-recognized antibodies for binding to CD26 also can be used.

In certain embodiments, an anti-CD26 antibody is used to determine CD26 expression. In some embodiments, an anti-CD26 antibody is selected for its ability to bind to CD26 in formalin-fixed, paraffin-embedded (FFPE) tissue specimens. In other embodiments, an anti-CD26 antibody is capable of binding to CD26 in frozen tissues. In further embodiments, an anti-CD26 antibody is capable of distinguishing membrane bound, cytoplasmic, and/or soluble forms of CD26.

### Immunosuppressive Agents

In some embodiments, the methods of the invention feature using one or more glucocorticoids and/or immunosuppressive agents in combination with the anti-CD26 antibody to treat dermatomyositis. In one embodiment, the immunosupressive agent is considered the standard of care for treatment of the dermatomyositis. An “immunosupressive agent” is a compound that inhibits or prevents the activity of the immune system. In one embodiment, immunosuppressant agent is methotrexate, azathioprine, or mycophenolate.

Glucocorticoids can be anti-inflammatory agents. As used herein, the term glucocorticoid can include but is not limited to methylprednisolone, prednisolone, dexamethasone, betamethasone, fluticasone propionate, budesonide, flunisolide, mometasone furoate, triamcinolone acetonide, rofleponide, ciclesonide, and butixocort propionate.

In one embodiment, the glucocorticoids or immunosuppressive agents include pharmaceutically acceptable salts, acids or derivatives of any of the above; as well as combinations of two or more of the above.

### Pharmaceutical Compositions

Pharmaceutical compositions suitable for administration to human patients are typically formulated for parenteral administration, e.g., in a liquid carrier, or suitable for reconstitution into liquid solution or suspension for intravenous administration.

In general, such compositions typically comprise a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions may be employed as carriers, particularly for injectable solutions (e.g., comprising an anti-CD26 antibody). Liquid compositions for parenteral administration can be formulated for administration by injection or continuous infusion. Routes of administration by injection or infusion include intravenous, intraperitoneal, intramuscular, intrathecal and subcutaneous. In one embodiment, the anti-CD26 antibody is administered intravenously.

### Methods of the Invention

In some embodiments, the present disclosure is directed to a method investigate the pharmacokinetics, pharmacodynamics, safety and clinical activity of an anti-CD26 antibody (e.g., begelomab) as an initial treatment of dermatomyositis in combination with standard glucocorticoid and/or immunosuppressant therapy. In some embodiments, the immunosuppressant therapy is administration of methotrexate, azathioprine, or mycophenolate. The study is designed to investigate the pharmacokinetics, pharmacodynamics, safety and clinical activity of an anti-CD26 antibody (e.g., begelomab) as an initial treatment of dermatomyositis in combination with standard glucocorticoid and/or immunosuppressant therapy.

### EXAMPLES

### Overall Study Design and Plan Description

### Study Design

This is a multicenter, randomized, double-blind, placebo-controlled, two-period, cross-over, phase II study, with the aim to assess the efficacy and safety of begelomab in combination with standard steroid and/or immunosuppressant therapy in the treatment of patients with dermatomyositis.

Twenty patients will be enrolled into 2 arms receiving glucocorticoids and/or immunosuppressant therapy (azathioprine, mycophenolate, methotrexate) in combination with begelomab or placebo. At the end of the first-treatment period, patients will cross over to the other treatment arm, regardless of disease condition. A wash-out of 3 months is planned between the two treatment-periods.

This is a 2×2 cross-over design trial. A wash-out of 3 months is planned between each treatment-period to avoid the carry-over effect from the first treatment to the second one.

At the end of the whole treatment period a 3 months follow-up is planned. Screening to confirm eligibility will take place not earlier than 1 week before the baseline visit (Day −7 to Day 0).

### Treatment Period

The experimental phase of this study will consist of two treatment periods of 1 month each spaced out by 3 months wash-out period.

Each treatment period will consist of a 5-days induction phase with daily administration of begelomab, followed by a maintenance phase with begelomab administration performed 3 times per week (every Mon, Wed, Fri) for a total of 11 administrations. Therefore, each treatment period will consist of 16 begelomab administrations (5 Induction and 11 Maintenance).

Wash-out period will separate the two treatment periods. At the beginning of the second and third month of Wash Out period (M2 and M3 from treatment start) safety assessment and disease evaluation will be carried out.

The follow up period will consist of 3 months at the end of the experimental phase. One visit per month is scheduled.

### Study Objectives

The primary objective of the trial will be:
to assess the efficacy of adding begelomab to glucocorticoid and/or immunosuppressant therapy (methotrexate, azathioprine, mycophenolate) compared with glucocorticoid and/or immunosuppressant plus placebo in the treatment of patients with dermatomyositis (DM).

The secondary objectives of the trial will be:
to assess the improvement of skin manifestations in begelomab arm compared to placebo;
to assess the safety and tolerability of multiple doses of begelomab in patients with dermatomyositis compared to placebo;
to assess if the treatment with begelomab is able to improve the quality of life in patients with dermatomyositis compared to placebo;
To assess if the treatment with begelomab is able to reduce or maintain a stable glucocorticoid/immunosuppressant therapy

The exploratory objective of the trial will be:
To assess if the treatment with begelomab is able to reduce dermatomyositis-relevant cytokines/chemokine levels (TNFα, IL-1β, IL-6, IL-17, IL-21, IFNγ, IFNα)

### Study Endpoints

The primary endpoint of the study will be to assess the mean difference (Begelomab vs. Placebo) in International Myositis Assessment & Clinical Studies Group (IMACS) total improvement score at Day 31 in each treatment period.

The secondary endpoints will be:
Number of subjects who achieve IMACS Definition of Improvement (IMACS DOI) at Day 31 in each treatment period in the begelomab arm compared to placebo.

### The IMACS DOI is:

An improvement of ≥20% from baseline in 3 IMACS core measures AND
No more than 2 IMACS core measure scores worsening by ≥25% from baseline, AND
Manual Muscle Test (MMT-8) may not decrease by ≥25% from baseline.
The change from baseline of Cutaneous Dermatomyositis Disease Area and Severity Index (CDASI) Activity Score at Day 31 in each treatment period and at M2/M6 in the begelomab arm compared to placebo.
Mean difference in International Myositis Assessment and Clinical Studies (IMACS) total improvement score at M2/M6 in the begelomab arm compared to placebo.
The change from baseline of HAQ (Health Assessment Questionnaire) or PGA (Patient Global Activity) score at Day 31 in each treatment period and at M2/M6 in the begelomab arm compared to placebo.
Number of subjects with a change (increase or taper) in glucocorticoid/immunosuppressant therapy (begelomab vs placebo).
Proportion of participants who have a treatment-related adverse event (TRAE) at Day 15 and Day 31 in each treatment period.
   The exploratory end-points will be (begelomab vs. placebo):
The change from baseline in DM-relevant cytokine/chemokine levels (TNFα, IL-1β, IL-6, IL-17, IL-21, IFNγ, IFNα) at Day 31 in each treatment period and at M2/M6 in the begelomab arm compared with placebo.

### Study Population

The study population will comprise patients higher than 18 years of age, irrespective of gender, who is diagnosed with DM.

Only patients who have signed the informed consent form and meet all inclusion criteria and no exclusion criteria will be included in the trial.

### Inclusion Criteria

To be eligible for inclusion into this study, each patient must fulfill ALL of the following inclusion criteria:
Written informed consent before starting any study-related procedure.
Males and Females aged ≥18 and ≤80 years old
Diagnosis of probable or definite DM according to Bohan and Peter criteria (1975) or ACR/EULAR criteria (Lundberg et al, 2017).
Although not mandatory, patients with muscle weakness are eligible for enrollment. Those with active muscle weakness must have a Manual Muscle Testing (MMT-8) score<142 out of 150.
Active skin disease as defined by a CDASI score≥5.
Stable glucocorticoid treatment for DM at a stable dose≤0.5 mg/kg methylprednisolone or prednisolone or equivalent for ≥4 weeks before randomization (Day 1).
Stable immunosuppressant treatment for DM for ≥4 weeks before randomization (Day 1), the stable treatment is defined as follows:
   Patients currently treated with oral or subcutaneous methotrexate (MTX) must have been on a stable dose of ≤25 mg per week.
   Patients currently treated with oral azathioprine (AZA) must have been on a stable dose of ≤3 mg/kg/day.
   Patients currently treated with oral mycophenolate (MMF) must have been on a stable dose of ≤3 g/day.
Patients that have received the following treatments can be enrolled only if the medications have been discontinued prior to screening visit:
   Rituximab: 9 months (Note: patients who received rituximab are only eligible for inclusion if B-cell counts are confirmed to be within normal limits)
   Intravenous immunoglobulin (IVIG): 3 months
Female subjects must meet one of the following criteria:
   Surgical sterilization (complete hysterectomy, bilateral tubal ligation and/or bilateral ovariectomy or tubal occlusion at least 6 months prior to first dosing), or have documented congenital sterility.
   Postmenopausal: defined as at least 12 months with no menses prior to screening and a serum follicle-stimulating hormone (FSH) to confirm postmenopausal status at screening.
   Women of child-bearing potential must agree to take adequate contraceptive measures in order to avoid any pregnancies during the course of the study (or for at least 3 months following the last dose of study drug, whichever is longer). Acceptable methods of birth control include oral, injected or implanted hormonal methods of contraception, placement of an intrauterine device (IUD) or intrauterine system (IUS), barrier methods of contraception: condom or occlusive cap (diaphragm or cervical/vault caps) with spermicidal foam/gel/film/cream/suppository. Abstinence is only considered an acceptable form of contraception when it is the usual life style of an individual.
Male patient who is surgically sterile (vasectomy), or male patient who is willing to agree with the true abstinence (refrain from heterosexual intercourse) or who uses barrier contraceptive measures during the entire study treatment period and for 3 months after the last administration of study drug. It is also acceptable where patient partner is making use of oral contraceptive instead.
Women must have a negative pregnancy test at Screening and at Baseline and must not be breastfeeding.
Subject must be willing and able to comply with study requirements, remain at the clinic, and return to the clinic for the follow-up evaluation, as specified in this protocol during the study period.

### Investigational Product

In this study, the Investigational Medicinal Product (IMP) will be begelomab, a murine monoclonal IgG2b antibody against CD26, produced in a hybridoma cell line.

Begelomab will be provided as a concentrated solution for i.v. infusion. The excipient present in begelomab is Dulbecco Phosphate Buffer Saline (DPBS). The composition is described in the Master formula in Table 1.

TABLE 1
Begelomab 2 mg/ml Master formula

| Component | Amount for 1 vial | | Function |
|---|---|---|---|
| Murine Monoclonal | 20 | mg | Drug Substance |
| antibody against CD 26 | | | |
| DPBS | | | Excipient |
| KCl | 2 | mg | |
| KH₂PO₄ | 2 | mg | |
| NaCl | 80 | mg | |
| Na₂HPO₄ x 7H₂O | 21.6 | mg | |
| Water for injection | to 10 | mL | |

The matching placebo will be identical to the study product with the exception of the murine monoclonal antibody begelomab.

A dose level of 16 mg/m² has been selected for this study, since PK/PD modelling from the ADN014 study in patients with acute Graft-vs-Host Disease (aGvHD) have demonstrated that this dose level provides near-maximal CD26 occupancy of CD26+ T-lymphocytes in the central compartment as well as a substantial (70-75%) suppression of soluble CD26 in serum. Systemic exposure at this dose level represents about 10% of the NOAEL exposure in non-clinical safety studies. The 16 mg/m² dose level has been safe and well tolerated by patients with aGvHD.

At 16 mg/m², once daily administration of begelomab is associated with a 5-6-fold serum accumulation from Day 1 to Day 5, while trough levels suggest that administration every other day corresponds to a steady state situation with no further serum accumulation. Interindividual variability in exposure is moderate and no obvious correlations between clearance and demographic or phenotypic modifiers have been observed. Data from the ADN014 study suggests that occupancy is closely linked to serum levels of begelomab, indicating relatively rapid on-and off-rates, requiring at least an every-other-day administration schedule to maintain occupancy and suppression of soluble CD26 levels.

During the Induction phase, patients will be administered with 16 mg/m² begelomab or the corresponding placebo once daily for five days (Days 1-5). During the Maintenance phase, patients will be administered with 16 mg/m² begelomab or the corresponding placebo three times per week (Mon, Wed, Fri) for another eleven doses (Days 8, 10, 12, 15, 17, 19, 22, 24, 26, 29, 31) for a total of 16 doses.

A wash-out period of 12 weeks will follow on day 32, after the end of the first treatment-period. Patients randomized to begelomab in the first Treatment Period will start placebo administration and patients randomized to placebo in the first Treatment Period will start begelomab 16 mg/m² once wash-out period is concluded. During second Treatment Period patients will continue the assigned treatment for 16 doses according to the scheme used in Treatment Period 1.

Placebo vials, identical in appearance and indistinguishable from the active treatment begelomab, will be used in the trial. Placebo will be administered in two periods at each patient according to the randomization list. Administration, compliance and accountability will be identical to the procedures applied to the active substance begelomab since this is a double-blind trial.

### Prior and Concomitant Therapies

Prior therapies refer to medication started within 4 weeks prior to screening and stopped prior to the first administration of the study treatment. Concomitant medication refers to medication started prior to and continued after the first administration of study treatment or taken any time after the first administration of the study treatment up to the follow-up visits.

Use of all concomitant medications or procedures will be recorded in the electronic data capture (EDC), regardless of whether they are permitted or prohibited, up to the end of the study (including the follow-up period).

Any concomitant medication deemed necessary for the welfare of the subject during the study may be given at the discretion of the investigator. However, it is the responsibility of the investigator to ensure that details regarding the medication are recorded in full in the EDC. The minimum requirement is that the drug name, dose, indication and the dates of administration are recorded. Any changes in concomitant medications will also be recorded in the EDC.

### Not Allowed Medications

The following medications are not allowed during the study (their use is a major deviation and should be documented as such) and concomitant use during screening will result in subject exclusion:
Systemic corticosteroid therapy for indication other than DM; the application of topical steroids is allowed.
No increase in the dosage of glucocorticoid therapy for DM (methylprednisolone, prednisolone or equivalent) above 0.2 mg/kg is allowed during the study: in case of the dosage is increased for any reason the patient will be withdrawn from the study.
Any agent for the treatment of DM other than begelomab or the stable immunosuppressant therapy (methotrexate, azathioprine and mycophenolate).
No increase in the dosage of the immunosuppressant therapy (methotrexate, azathioprine and mycophenolate) is allowed during the study above the dosage permitted at study entry: in case of the dosage is increased for any reason above that threshold the patient will be withdrawn from the study.
Any other investigational product from 4 weeks prior to enrolment and during the study participation.

### Allowed Medications

The following medications are allowed during the study:
Glucocorticoids treatment for DM at a stable dose of no more/equal to 0.2 mg/kg methylprednisolone, prednisolone or equivalent. No increase in the dosage of glucocorticoid therapy above this concentration is allowed during the study.
Concurrent use or addition of topical steroid therapy (skin creams, inhaled beclomethasone, and other non-absorbable steroids) is allowed.
Stable immunosuppressant treatment for DM defined as follows:
   Oral or subcutaneous methotrexate at a stable dose of ≤25 mg per week.
   Oral azathioprine at a stable dose of ≤3 mg/kg/day.
   Oral mycophenolate at a stable dose of ≤3 gr/day.
No increase in the dosage of immunosuppressant therapy above this concentration is allowed during the study.

### Sequences

SEQ ID NO: 1 Heavy Chain Constant Region Amino Acid Sequence; Anti-CD26 mAb
(begelomab)
AKTTPPSVYPLAPGCGDTTGSSVTLGCLVKGYFPESVTVTWNSGSLSSSVHTFPALLQSG
LYTMSSSVTVPSSTWPSQTVTCSVAHPASSTTVDKKLEPSGPISTINPCPPCKECHKCPA
PNLEGGPSVFIFPPNIKDVLMISLTPKVTCVVVDVSEDDPDVQISWFVNNVEVHTAQTQT
HREDYNSTIRVVSTLPIQHQDWMSGKEFKCKVNNKDLPSPIERTISKIKGLVRAPQVYIL
PPPAEQLSRKDVSLTCLVVGFNPGDISVEWTSNGHTEENYKDTAPVLDSDGSYFIYSKLN
MKTSKWEKTDSFSCNVRHEGLKNYYLKKTISRSPGK
SEQ ID NO: 2 Light Chain Constant Region Amino Acid Sequence; Anti-CD26 mAb (begelomab)
RADAAPTVSIFPPSSEQLTSGGASVVCFLNNFYPKDINVKWKIDGSERQNGVLNSWTDQD
SKDSTYSMSSTLTLTKDEYERHNSYTCEATHKTSTSPIVKSFNRNEC
SEQ ID NO: 3 Heavy Chain Variable Region (VH) Amino Acid Sequence; Anti-CD26 mAb
(begelomab)
QVQLQQSGAELVKPGASVKLSCKASGYTFRSYDINWVRQRPEQGLEWIGWIFPGDGSTKY
NEKFKGKATLTTDKSSSTAYMQLSRLTSEDSAVYFCARWTVVGPGYFDVWGAGTTVTVSS
SEQ ID NO: 4 Heavy Chain Variable Region (VH) Nucleotide Sequence; Anti-CD26 mAb
(begelomab)
caggtccagctgcagcagtctggagctgaactggtaaagcctggggcttcagtgaagttgtcctg
caaggcttctggctacaccttcagaagttatgatataaactgggtgagacagaggcctgaacagg
gacttgagtggattggatggatttttcctggagatggtagtactaagtacaatgagaagttcaag
ggcaaggccacactgactacagacaaatcctccagcacagcctacatgcagctcagcaggctgac
atctgaggactctgctgtctatttctgtgcaagatggacggtagtaggcccagggtacttcgatg
tctggggcgcagggaccacggtcaccgtctcctca
SEQ ID NO: 5 Light Chain Variable Region (VL) Amino Acid Sequence; Anti-CD26 mAb
(begelomab)
QIVLTQSPAIMSASPGEKVTITCSASSSVSYMNWFQQKPGTSPKLWIYSTSNLASGVPAR
FSGSGSGTSYSLTISRMEAEDAATYYCQQRSSYPNTFGGGTKLEIK
SEQ ID NO: 6 Light Chain Variable Region (VL) Nucleotide Sequence; Anti-CD26 mAb
(begelomab)
Caaattgttctcacccagtctccagcaatcatgtctgcatctccaggggagaaggtcaccataac
ctgcagtgccagctcaagtgtaagttacatgaactggttccagcagaagccaggcacttctccca
aactctggatttatagcacctccaacctggcttctggagtccctgctcgcttcagtggcagtgga
tctgggacctcttactctctcacaatcagccgaatggaggctgaagatgctgccacttattactg
ccagcaaaggagtagttacccgaacacgttcggaggggggaccaagctggaaataaaa
SEQ ID NO: 7 Heavy Chain CDR1 Amino Acid Sequence; Anti-CD26 mAb (begelomab)
GYTFRSYDIN
SEQ ID NO: 8 Heavy Chain CDR2 Amino Acid Sequence; Anti-CD26 mAb (begelomab)
WIFPGDGSTKYNEKFK
SEQ ID NO: 9 Heavy Chain CDR3 Amino Acid Sequence; Anti-CD26 mAb (begelomab)
WTVVGPGYFDV
SEQ ID NO: 10 Light Chain CDR1 Amino Acid Sequence; Anti-CD26 mAb (begelomab)
SASSSVSYMN
SEQ ID NO: 11 Light Chain CDR2 Amino Acid Sequence; Anti-CD26 mAb (begelomab)
STSNLAS
SEQ ID NO: 12 Light Chain CDR3 Amino Acid Sequence; Anti-CD26 mAb (begelomab)
QQRSSYPNT
SEQ ID NO: 13 Heavy Chain Constant Region Nucleotide Sequence; Anti-CD26 mAb
(begelomab)
gccaaaacaacacccccatcagtctatccactggcccctgggtgtggagatacaactggttcctc
cgtgactctgggatgcctggtcaagggctacttccctgagtcagtgactgtgacttggaactctg
gatccctgtccagcagtgtgcacaccttcccagctctcctgcagtctggactctacactatgagc
agctcagtgactgtcccctccagcacctggccaagtcagaccgtcacctgcagcgttgctcaccc
agccagcagcaccacggtggacaaaaaacttgagcccagcgggcccatttcaacaatcaacccct
gtcctccatgcaaggagtgtcacaaatgcccagctcctaacctcgagggtggaccatccgtcttc
atcttccctccaaatatcaaggatgtactcatgatctccctgacacccaaggtcacgtgtgtggt
ggtggatgtgagcgaggatgacccagacgtccagatcagctggtttgtgaacaacgtggaagtac
acacagctcagacacaaacccatagagaggattacaacagtactatccgggtggtcagcaccctc
cccatccagcaccaggactggatgagtggcaaggagttcaaatgcaaggtcaacaacaaagacct
cccatcacccatcgagagaaccatctcaaaaattaaagggctagtcagagctccacaagtataca
tcttgccgccaccagcagagcagttgtccaggaaagatgtcagtctcacttgcctggtcgtgggc
ttcaaccctggagacatcagtgtggagtggaccagcaatgggcatacagaggagaactacaagga
caccgcaccagtcctggactctgacggttcttacttcatatatagcaagctcaatatgaaaacaa
gcaagtgggagaaaacagattccttctcatgcaacgtgagacacgagggtctgaaaaattactac
ctgaagaagaccatctcccggtctccgggtaaa
SEQ ID NO: 14 Light Chain Constant Nucleotide Sequence; Anti-CD26 mAb (begelomab)
cgggctgatgctgcaccaactgtatccatcttcccaccatccagtgagcagttaacatctggagg
tgcctcagtcgtgtgcttcttgaacaacttctaccccaaagacatcaatgtcaagtggaagattg
atggcagtgaacgacaaaatggcgtcctgaacagttggactgatcaggacagcaaagacagcacc
tacagcatgagcagcaccctcacgttgaccaaggacgagtatgaacgacataacagttatacctg
tgaggccactcacaagacatcaacttcacccattgtcaagagcttcaacaggaatgagtgt
SEQ ID NO: 15 Heavy Chain Nucleotide Sequence; Anti-CD26 mAb (begelomab-CHO optimized)
caagtgcagcttcagcagtccggagccgaactcgtgaagccgggagcttccgtgaagctgagctg
caaggcatcggggtataccttccgctcctacgacatcaactgggtcagacagaggcccgaacagg
gtctggaatggattggctggatcttccctggcgacggttccaccaagtacaacgagaagttcaag
ggcaaagccaccctgaccactgacaagtcctcatcgaccgcgtacatgcaattgagccggctgac
ctccgaggatagcgccgtgtacttctgtgcccggtggactgtcgtgggaccaggctactttgatg
tctggggggccggaactaccgtgacggtgtcatcagccaagactacccctccgtccgtgtacccc
cttgctccgggatgtggagacaccaccggctcgtccgtcactctgggatgcctcgtgaagggata
cttccccgaatccgtcaccgtgacctggaacagcggaagcctgtcctcgtccgtgcatactttcc
ctgccctgctgcaatccggcctgtacaccatgagctccagcgtgaccgtgccatcctcgacctgg
cccagccagaccgtgacttgctcagtggcgcaccctgcctcatccactaccgtggacaagaagct
cgagccctccggtccgatttcaaccatcaacccttgcccaccctgcaaagaatgccataagtgtc
ccgctccgaatctagaaggcggcccatccgtctttatcttccctcccaacattaaggacgtgctg
atgattagtctgaccccgaaagtcacttgcgtggtggtggacgtgtccgaagatgacccagacgt
gcagatctcatggttcgtgaacaacgtggaggtgcacacggcccagacccagacgcaccgggagg
actacaactcgactatccgcgtggtgtccacccttccgatccaacaccaggattggatgtcgggg
aaggagttcaagtgcaaggtcaacaacaaggatctcccgtcccccattgagaggacaatctctaa
gatcaagggcctcgtcagagcgcctcaggtctacatcttgcctcctcccgccgaacagttgagcc
ggaaggatgtgtccctgacttgtctggtcgtggggttcaatccgggagacatctccgtggagtgg
acctcgaacggacacaccgaggaaaactacaaggacactgcaccggtgctggattccgacggctc
ctatttcatctactcgaagctgaacatgaaaacctcgaaatgggaaaagactgacagcttcagct
gcaacgtgcgccacgagggtctgaagaactactacctgaaaaagaccatttcacggtccccgggg
aaa
SEQ ID NO: 16 Light Chain Nucleotide Sequence; Anti-CD26 mAb (begelomab-CHO optimized)
Cagatcgtgcttacccaatccccggcgattatgtcagccagccccggagaaaaggtcaccattac
ttgctcggcatcctcctccgtgtcgtacatgaactggttccagcaaaagcccggcactagcccaa
agctgtggatctattccacgtccaacctggcgtcaggagtgcctgcccgcttttcgggttctggc
agcgggactagctactccctcaccatctcgagaatggaagctgaggacgccgccacctactactg
tcagcagcggtcctcctacccgaacaccttcgggggaggcaccaaactggagatcaaacgggctg
atgctgcaccaactgtatccatcttcccaccatccagtgagcagttaacatctggaggtgcctca
gtcgtgtgcttcttgaacaacttctaccccaaagacatcaatgtcaagtggaagattgatggcag
tgaacgacaaaatggcgtcctgaacagttggactgatcaggacagcaaagacagcacctacagca
tgagcagcaccctcacgttgaccaaggacgagtatgaacgacataacagctatacctgtgaggcc
actcacaagacatcaacttcacccatcgtcaagagcttcaacaggaatgagtgt

1. A method of treating dermatomyositis in a subject, comprising administering a therapeutically effective amount of an anti-CD26 antibody to the subject, wherein the antibody is administered at a dose of 16 mg/m²/day, and wherein the antibody is administered once daily for five days followed by administration three times per week for a total of 16 doses.

2. A method of reducing the levels of dermatomyositis-related cytokines in a subject, comprising administering a therapeutically effective amount of an anti-CD26 antibody to the subject, wherein the antibody is administered at a dose of 16 mg/m²/day, and wherein the antibody is administered once daily for five days followed by administration three times per week for a total of 16 doses.

3. The method of claim 2, wherein the dermatomyositis-related cytokines are tumor necrosis factor-alpha (TNFα), interleukin-1b (IL-1β) interleukin-6 (IL-6), interleukin-17 (IL-17), interleukin-21 (IL 21) interferon-alpha (IFNα) or interferon-gamma (IFNγ).

4. The method of claim 1, wherein the anti-CD26 antibody is a full-length antibody.

5. The method of claim 1, wherein the antibody is a monoclonal, human, humanized, chimeric, multivalent antibody, or an antigen-binding fragment thereof.

6. The method of claim 1, wherein the antibody has an isotype selected from the group consisting of IgG1, IgG2, IgG3, IgG4, IgM, IgA, IgD and IgE.

7. The method of claim 6, wherein the antibody has an IgG2b isotype.

8. The method of claim 1, wherein the anti-CD26 antibody is begelomab, IF7, or CM03.

9. The method of claim 8, wherein the anti-CD26 antibody is produced in Chinese hamster ovary (CHO) cells.

10. The method of claim 1, wherein the anti-CD26 antibody is produced from a hybridoma cell line deposited at CBA-ICLC of Genoa (Italy) as deposit number PD 12002.

11. The method of claim 1, wherein the anti-CD26 antibody comprises:
(a) a heavy chain variable region CDR1 comprising the sequence set forth in SEQ ID NO:7;
(b) a heavy chain variable region CDR2 comprising the sequence set forth in SEQ ID NO:8;
(c) a heavy chain variable region CDR3 comprising the sequence set forth in SEQ ID NO:9;
(d) a light chain variable region CDR1 comprising the sequence set forth in SEQ ID NO: 10;
(e) a light chain variable region CDR2 comprising the sequence set forth in SEQ ID NO: 11; and
(f) a light chain variable region CDR3 comprising the sequence set forth in SEQ ID NO: 12.

12. The method of claim 1, wherein the anti-CD26 antibody comprises heavy and light chain variable regions comprising the sequences set forth in SEQ ID NOs:3 and 5, respectively.

13. The method of claim 1, wherein the anti-CD26 antibody comprises heavy and light chains comprising the sequences set forth in SEQ ID NOs: 1 and 2, respectively.

14. The method of claim 1, further comprising administering a glucocorticoid and/or immunosuppressant therapy.

15. The method of claim 14, wherein the immunosuppressant therapy comprises administration of methotrexate, azathioprine, or mycophenolate.
